(19) United States
(12) Patent Application Publication
(10) Pub. No.: US 2023/0116808 A1
(43) Pub. Date: Apr. 13, 2023

(54) INFORMATION PROCESSING DEVICE AND PROGRAM

(71) Applicant: Konica Minolta, Inc., Tokyo (JP)

(72) Inventor: KAZUKI HARADA, Kobe-shi (JP)

(21) Appl. No.: 17/956,161

(22) Filed: Sep. 29, 2022

(30) Foreign Application Priority Data
Oct. 8, 2021 (JP) 2021-166197

(51) Int. Cl. A61B 5/16; G10L 25/63; G10L 15/18; G10L 15/26; G06V 40/16; G06V 40/18; G06V 40/20

(52) U.S. Cl. CPC A61B 5/165; G10L 25/63; G10L 15/1807; G10L 15/26; G06V 40/174; G06V 40/171; G06V 40/193; G06V 40/28

(57) ABSTRACT

An information processing device that determines a psychological state of a member in a meeting, includes: a voice processor that extracts a predetermined feature amount from a voice of a part corresponding to a speech of the member among voices collected during the meeting by a microphone; an estimator that receives an input of a feature amount and estimates a likelihood that the member is in a predetermined psychological state; and a determiner that determines a likelihood that the member is in the predetermined psychological state during the meeting on a basis of the likelihood estimated by the estimator and an index value according to a purpose of the meeting.

The entire disclosure of Japanese patent Application No. 2021-166197, filed on Oct. 8, 2021, is incorporated herein by reference in its entirety.

### BACKGROUND

### Technological Field

The present disclosure relates to an information processing device, and more particularly relates to an information processing device and a program for evaluating a psychological state of a meeting member.

### Description of the Related Art

There is a demand for efficiently conducting a meeting or an arrangement. For example, a meeting support system of JP 2017-215931 A evaluates content of a speech of a participant of a meeting. In addition, a system of JP 2021-86385 A determines a degree of achievement of a meeting policy. A meeting support system of JP 2020-154673 A determines a reacting state of another speech with respect to a speech, and evaluates the speech based on a determination result. A meeting support system disclosed of JP 2017-112545 A determines a degree of convergence of a discussion from voices of meeting participants.

In efficient management of a meeting, there are demands for obtaining support information based on a psychological state of a member according to the purpose of the meeting in order to support the management, but in JP 2017-215931 A, JP 2021-86385 A, JP 2020-154673 A, and JP 2017-112545 A described above, a configuration considering the psychological state of a meeting participant is not proposed.

### SUMMARY

One object of the present disclosure is to provide an information processing device and a program capable of determining a psychological state of a member of a meeting according to a purpose of the meeting.

To achieve the abovementioned object, according to an aspect of the present invention, there is provided an information processing device that determines a psychological state of a member in a meeting, and the information processing device reflecting one aspect of the present invention comprises: a voice processor that extracts a predetermined feature amount from a voice of a part corresponding to a speech of the member among voices collected during the meeting by a microphone; an estimator that receives an input of a feature amount and estimates a likelihood that the member is in a predetermined psychological state; and a determiner that determines a likelihood that the member is in the predetermined psychological state during the meeting on a basis of the likelihood estimated by the estimator and an index value according to a purpose of the meeting.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a schematic configuration of a meeting system according to the present embodiment;

FIG. 2 is a diagram illustrating a hardware configuration of a terminal according to the present embodiment;

FIG. 3 is a diagram illustrating a hardware configuration of an information processing device according to the present embodiment;

FIG. 4 is a diagram schematically illustrating a software configuration of a psychological state evaluation unit according to the present embodiment;

FIG. 5 is a diagram schematically illustrating an example of a determination index value DB according to the present embodiment;

FIG. 6 is a diagram illustrating an example of an estimation value according to the present embodiment;

FIGS. 7A to 7C are diagrams illustrating a determination method of a determiner according to the present embodiment;

FIG. 8 is a diagram schematically illustrating an example of a priority item management DB according to the present embodiment;

FIG. 9 is a diagram illustrating an example of a determination result using the priority item management DB of the determiner according to the present embodiment;

FIG. 10 is a diagram schematically illustrating an example of a report according to the present embodiment;

FIG. 11 is a diagram illustrating another configuration example of the determination index value DB according to the present embodiment;

FIG. 12 is a diagram illustrating an example of indexes before and after change in association with each other;

FIG. 13 is a diagram illustrating an example of indexes before and after change in association with each other;

FIG. 14 is a schematic flowchart of overall processing according to the present embodiment;

FIG. 15 is a flowchart of a routine in FIG. 14;

FIG. 16 is a flowchart of a routine in FIG. 15;

FIG. 17 is a flowchart of a routine in FIG. 15;

FIG. 18 is a flowchart of a routine in FIG. 14;

FIG. 19 is a flowchart of a routine in FIG. 18;

FIG. 20 is a flowchart of a routine in FIG. 14;

FIG. 21 is a flowchart of a feedback processing routine in FIG. 20;

FIG. 22 is a flowchart of a comparison routine of result information of a meeting in FIG. 20;

FIG. 23 is a flowchart of a recalculation processing routine of FIG. 22;

FIG. 24 is a diagram illustrating an example of a display screen of a display according to the present embodiment;

FIG. 25 is a diagram illustrating an example of a display screen of the display according to the present embodiment;

FIG. 26 is a diagram illustrating an example of a display screen of the display according to the present embodiment;

FIG. 27 is a diagram illustrating an example of a display screen of the display according to the present embodiment;

FIG. 28 is a diagram illustrating an example of a display screen of the display according to the present embodiment; and

FIG. 29 is a diagram illustrating a method of psychological state determination according to the present embodiment.

### DETAILED DESCRIPTION OF EMBODIMENTS

Hereinafter, one or more embodiments of the present invention will be described with reference to the drawings. However, the scope of the invention is not limited to the disclosed embodiments. In the following description, the same parts and components are denoted by the same reference numerals. Their names and functions are the same. Therefore, detailed descriptions thereof will not be repeated. Note that the embodiments and modification examples described below may be selectively combined as appropriate.

<A. Assessment of Psychological State>

In the present embodiment, a tool for evaluating a psychological state of a member participating in a meeting according to a purpose of the meeting is provided. Here, for the purpose of activating discussion and improving communication in companies, and the like, as a method of reducing a psychological barrier during an arrangement, a method focusing on “psychological safety” among psychological states has been proposed. Here, the psychological safety refers to a state in which psychological barriers of members are reduced in a meeting (for example, a state of being confident that other members do not reject his or her speech or do not impose a punishment). As an item for measuring the psychological safety, “7 questions” by Professor Edmonson is well known. Also in the present embodiment, the following “seven questions” for evaluating the psychological safety are used as items for evaluating the psychological states of the members of the meeting, but the items for evaluating the psychological safety are not limited thereto. In addition, the psychological state evaluated at the meeting is not limited to the psychological safety.

(a) Inventor's Experiment

The inventor conducted an experiment to measure an estimation value of high (or low) likelihood corresponding to each of the seven questions. The “high or low likelihood” indicates whether the psychological state of a person is likely or unlikely to be the psychological state (predetermined state) indicated in a question (item) of response. In the experiment, for example, a third party who is not informed of the purpose of the meeting performs the measurement on the basis of an observation result of the speech of a meeting member. In addition, the “estimation value” is a value quantitatively indicating that “lower likelihood is better” or “higher likelihood is better” or the like. Hereinafter, this “question” is also referred to as an “item” for determining the psychological state. Note that the “team” in the question can be replaced with “meeting”.

Question (1) If a mistake is made on this team, it is often criticized (lower likelihood is better);

Question (2) Members of this team are able to bring up problems and negative issues (higher likelihood is better);

Question (3) Members on this team sometimes tend to reject others for being different (lower likelihood is better);

Question (4) It feels safe to take a risk on this team (higher likelihood is better);

Question (5) It is difficult to ask other members of this team for help (lower likelihood is better);

Question (6) No member on this team would deliberately act in a way that undermines my efforts (higher likelihood is better); and

Question (7) When advancing work in the current team, it feels that my skills are exhibited (higher likelihood is better).

(a1. Evaluation of Estimation Value Based on Purpose of Meeting)

The estimation value of likelihood described above may vary for the same item depending on the purpose of the meeting. Specific examples will be described by exemplifying the following experiments of Case 1 and Case 2.

(Case 1: Brainstorming Meeting for Idea Development, New Function Development, or the Like)

(A) In a case where the meeting is a brainstorming meeting for idea development, new function development, or the like, when members make a productive speech without making a negative speech in accordance with the purpose (rules) of the meeting, a third party other than the members observes the content of the speeches in the meeting for the questions (1) to (3), and performs conversion into scores with respect to the determination of the likelihood of each question on the basis of observation results. In this case, the following estimation values converted into scores were obtained.

Question (1) If a mistake is made on this team, it is often criticized (lower likelihood is better)→10 points;

Question (2) Members of this team are able to bring up problems and negative issues (higher likelihood is better)→0 points;

Question (3) Members on this team sometimes tend to reject others for being different (higher likelihood is better)→10 points.

In this meeting, since the members make a speech in accordance with the purpose of the meeting, observation results of the third party show that there is no critical or negative speech and there is a high tendency to accept a different speech. Therefore, since it is observed that there is no negative speech in the question (2), the estimation value converted into scores is 0 points. Consequently, the questions (1) to (3) are 20 points in total.

(B) In a case where the meeting is the brainstorming meeting for idea development, new function development, or the like, when members make many negative speeches and few productive speeches contrary to the purpose (rules) of the meeting, the third party other than the members observes the content of the speeches of the meeting for the questions (1) to (3), and performs conversion into scores with respect to the determination of the likelihood of each question on the basis of observation results. In this case, the following estimation values were obtained.

Question (1) If a mistake is made on this team, it is often criticized (lower likelihood is better)→10 points;

Question (2) Members of this team are able to bring up problems and negative issues (higher likelihood is better)→10 points;

Question (3) Members on this team sometimes tend to reject others for being different (higher likelihood is better)→10 points.

In this meeting, since there are many speeches contrary to the purpose of the meeting, the observation results of the third party are such that there are many negative speeches and the members quarrel with each other making the question (1) indicate 10 points, the negative speeches are determined to be courageous and active discussion making the question (2) indicate 10 points, and there are many speeches and thus it is determined that there is a high tendency to accept different speeches making the question (3) indicate 10 points. Consequently, the questions (1) to (3) are 30 points in total.

According to the experiment, the members who participated in both the meetings of the cases (A) and (B) were in a psychological state that they are not satisfied that the evaluation (total estimation value) of the observer was higher in the case (B) where the speech is made contrary to the purpose of the meeting than in the case (A) where the speech is made according to the purpose of the meeting, and the members who participated only in the meeting of the case (B) were in a psychological state that they were difficult to make a speech because a speech is to be negated.

According to these experiments, in a case where the likelihood determination of each item is quantitatively converted into scores, the score of the likelihood determination deviates from the actual psychological states of the members of the meeting because it is not possible to perform the conversion into scores based on the purpose (rule) of the meeting.

(Case 2: Progress Meeting and Review Meeting)

The likelihood assessment was evaluated for a progress meeting in which the progress status of the business was shared or a review meeting in which the content of the implemented business was reviewed. More specifically, in a case where a member fails due to a trivial mistake in work, when the member speaks the same content at a meeting for different purposes, the meaning of the speech and the way of interpreting the speech for the member change in consideration of the purpose of the meeting.

In the experiment, the psychological states of other members were observed when a member at a meeting said “The mistake is a problem. Why did you make such a mistake at this time. Has check been done?”

For example, when the meeting is a progress meeting, in a case where a certain member makes the abovementioned speech, the member has an attentive and critical psychological state, and in response to the speech, other members are in a psychological state of “I was blamed for the mistake, and it is difficult to report the mistake next time,” and tend to make fewer speeches. On the other hand, in the review meeting, in a case where a certain member makes the above speech, the member has a psychological state for checking and improvement (productive step) to prevent the mistake, and in response to the speech, other members have a psychological state of rechecking the mistake in order to prevent the recurrence of the mistake.

With respect to the speeches at the progress meeting and the review meeting, regarding the questions (1) to (3), the likelihood of each question is converted into scores on the basis of the observation result such as the contents of the speeches at the meeting by the third party. In this case, the following experimental results were obtained.

Question (1) If a mistake is made on this team, it is often criticized (lower likelihood is better)→20 points;

Question (2) Members of this team are able to bring up problems and negative issues (higher likelihood is better)→10 points;

Question (3) Members on this team sometimes tend to reject others for being different (higher likelihood is better)→5 points.

In each of these two meetings, the speeches of a certain member are critical speeches and the question (1) has 20 points, the above speeches have negative contents and thus the question (2) has 20 points, and the question (3) has 5 points since some opinions are present.

However, since a certain member of the review meeting, that is, the reviewer, has a high score of the question (1) although the above speech is not critical, this member feels that his or her psychological state deviates from the estimation value of the likelihood determination of each item.

Therefore, even in this experiment, since it is not possible to perform the conversion of the determination into scores based on the purpose (rule) of the meeting, a result that the estimation value deviates from the actual psychological states of the members of the meeting was obtained.

On the basis of the results of such an experiment, the inventor has obtained knowledge that, in a case where the psychological safety is determined by the system, when an item (question) is set and a determination is made for the item, the final likelihood has to be determined on the basis of the purpose of the meeting in order to prevent deviation of the determination from the psychological state of the member.

On the basis of this knowledge, the inventor has also obtained knowledge that it is possible to determine the likelihood of the psychological state according to the purpose of the meeting by setting weighting according to the purpose to each item for each type of the meeting, that is, for each purpose of the meeting, and evaluating an estimation value of the likelihood of each item by weighting the item for each purpose of the meeting. This weighting is hereinafter also referred to as an index value. Hereinafter, a meeting support system based on these pieces of knowledge will be described.

Note that, in the present embodiment, a meeting is exemplified as a scene where a plurality of members speaks to each other. The meeting of the present embodiment is a concept including arrangement, meeting, consultation, and the like.

<B. Meeting Support System>

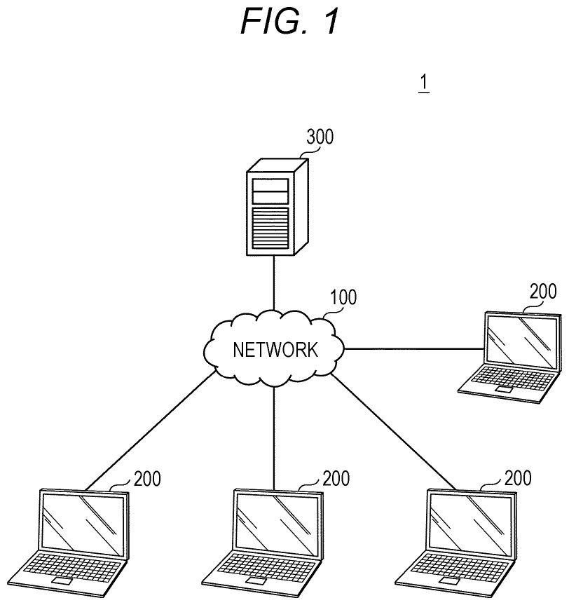

FIG. 1 is a diagram illustrating a schematic configuration of a meeting system according to the present embodiment. Referring to FIG. 1, a meeting system 1 is a system for providing an online meeting that exchanges images (videos) and voices via a network, and includes a plurality of terminals 200 that can be operated by members (participants) participating in the meeting, and an information processing device 300 that communicates with the plurality of terminals 200 via the network 100. Hereinafter, the online meeting is abbreviated as a “meeting”.

The terminals 200 each includes, for example, a general-purpose personal computer (PC), a smartphone, a tablet, or the like. The information processing device 300 provides a platform for achieving a meeting system as a cloud server having the terminal 200 as a client, for example. The network 100 may be, for example, a public network such as the Internet, a public line, or a public wireless local area network (LAN), or may be a private network such as a LAN or a virtual private network (VPN). The information processing device 300 has been exemplified as a cloud server, but is not limited thereto, and may be an on-premises server.

The information processing device 300 supports online meetings among the plurality of terminals 200. For example, the information processing device 300 receives video data, voice data, and the like transmitted from one terminal 200, and transfers the received video data or voice data to the other terminal 200. Thus, the members can share video and voice by operating the terminal 200.

In addition, the information processing device 300 evaluates the psychological state of a member during the meeting for each item according to the purpose of the meeting from voice data or image data exchanged between the terminals 200 in the meeting, and outputs an evaluation result.

(b1. Hardware Configuration)

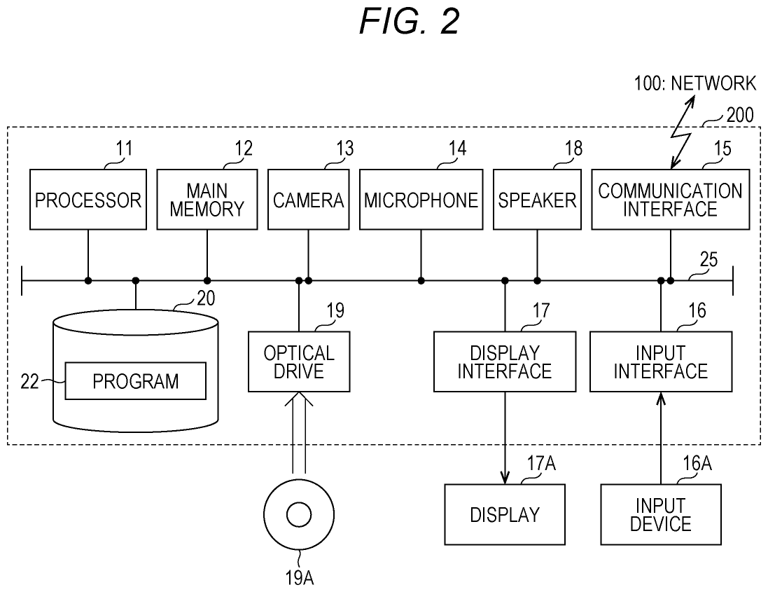

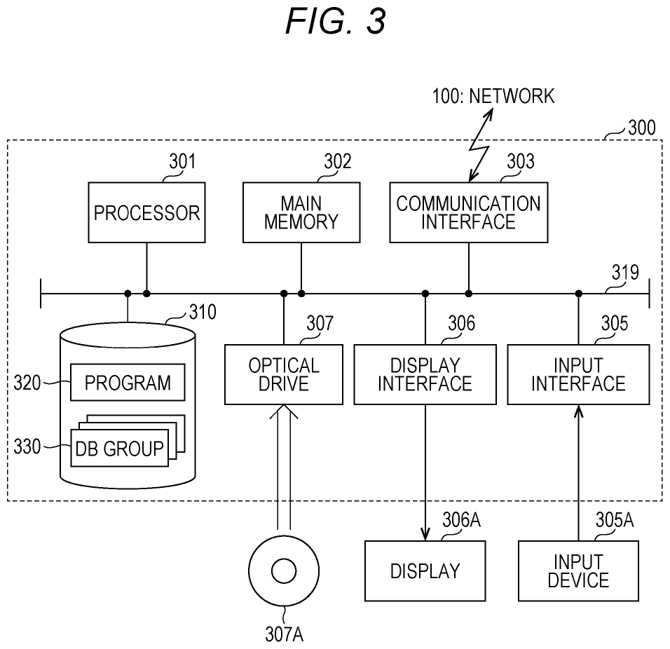

FIG. 2 is a diagram illustrating a hardware configuration of the terminal 200 according to the present embodiment. FIG. 3 is a diagram illustrating a hardware configuration of the information processing device 300 according to the present embodiment.

Referring to FIG. 2, the terminal 200 includes a processor 11, a main memory 12 including a volatile storage medium including a read only memory (ROM), a random access memory (RAM), and the like, a camera 13, a microphone 14, a communication interface 15 including a communication circuit including a network interface card (NIC), an input interface 16 to which an input device 16A such as a keyboard or a mouse operable by a user is connected, a display interface 17 to which a display 17A is connected, a speaker 18, an optical drive 19 to which an optical disk 19A as an example of a recording medium is detachably mounted, and a storage device 20 including a nonvolatile recording medium such as a hard disk drive (HDD) or a solid state drive (SSD). These components are communicably connected to each other via an internal bus 25. The input interface 16 receives a user operation on the terminal 200 via the input device 16A. In addition, the display interface 17 includes a driver circuit that drives the display 17A on the basis of image data so as to display an image. The storage device 20 stores a program 22 that implements various processes when read and executed by the processor 11.

The processor 11 includes, for example, at least one integrated circuit. The integrated circuit includes, for example, at least one central processing unit (CPU), at least one application specific integrated circuit (ASIC), at least one field programmable gate array (FPGA), or a combination thereof or the like.

The processor 11 develops the program 22 stored in the storage device 20 in the main memory 12 and executes the program, thereby implementing various processes according to the present embodiment. The main memory 12 provides a work memory necessary for execution of a program by the processor 11.

The camera 13 is an imaging device including a complementary metal oxide semiconductor (CMOS) sensor, a charge coupled device (CCD) sensor, or the like. The imaging visual field of the camera 13 has a range in which a member or the like who operates the terminal 200 can be imaged, and generates image data such as a captured moving image (video) or still image. The microphone 14 is configured to collect voice around the terminal 200. The voice collected by the microphone 14 may include a voice or the like of a member who operates the terminal 200.

The communication interface 15 exchanges data and signals with the information processing device 300 via the network 100. As an example, the terminal 200 transmits and receives image data, voice data, and the like to and from the information processing device 300 via the communication interface 15. The image data includes image data based on a captured image of the camera 13 of the terminal 200, and the voice data includes voice data collected by the speaker 18 of the terminal 200.

During the meeting, the information processing device 300 receives the image data and the voice data transmitted from the terminal 200, and transfers the received image data and voice data to the terminal 200 of another member. The terminal 200 displays an image based on the image data received from the information processing device 300 on the display 17A, and outputs a voice based on the voice data received from the information processing device 300 from the speaker 18. Thus, during the meeting, the members can communicate by exchanging images and voices with each other via the terminal 200.

Furthermore, the communication interface 15 can also receive screen data from the information processing device 300. More specifically, the information processing device 300 transmits a web page to the terminal 200 as a web server, and a web browser of the terminal 200 displays a screen based on the web page from the information processing device 300 on the display 17A via the display interface 17.

From the optical disk 19A or the like, which is an external storage medium, the optical drive 19 reads various programs (for example, the program 22) stored therein and installs the programs in the storage device 20. FIG. 2 illustrates a configuration example in which a necessary program is installed in the terminal 200 from an external storage medium, but the present invention is not limited thereto, and the program may be downloaded from another device or the like on the network 100. In addition, the external storage medium is not limited to the optical disk 19A, and may be, for example, a storage medium such as a universal serial bus (USB) memory, a secure digital (SD) card, or a compact flash (CF).

The program 22 of the storage device 20 may be provided not as a single program but by being incorporated in a part of an arbitrary program. In this case, the processing according to the present embodiment is achieved in cooperation with an arbitrary program. Even a program not including such a part of modules does not depart from the gist of terminal 200 according to the present embodiment. In addition, a part or all of the functions provided by the program 22 may be achieved by dedicated hardware. In addition, the camera 13, the microphone 14, and the speaker 18 may be externally attached to the terminal 200, and the number of these devices is not limited to one and a plurality of these devices may be connected to the terminal 200.

Referring to FIG. 3, the information processing device 300 includes a processor 301, a main memory 302 including a volatile storage medium including a ROM, a RAM, and the like, a communication interface 303 including a communication circuit including an NIC, an input interface 305 to which an input device 305A such as a keyboard or a mouse operable by a user is connected, a display interface 306 to which a display 306A is connected, an optical drive 307 to which an optical disk 307A as an example of a recording medium is detachably mounted, and a storage device 310 including a nonvolatile recording medium such as an HDD or an SSD. These components are communicably connected to each other via an internal bus 319. The input interface 305 receives a user operation on the information processing device 300 via the input device 305A. Furthermore, the display interface 306 includes a driver circuit that drives the display 306A on the basis of image data so as to display an image. The storage device 310 stores software read and executed by the processor 11. The software includes the program 22 that achieves various processes and a DB group 330 including a plurality of databases (DBs).

The processor 301 includes, for example, at least one integrated circuit. The integrated circuit includes, for example, at least one CPU, at least one ASIC, at least one FPGA, or a combination thereof or the like.

The processor 301 develops a program 320 stored in the storage device 310 in the main memory 302 and executes the program, thereby implementing various processes according to the present embodiment. The main memory 302 provides a work memory necessary for execution of a program by the processor 301.

The communication interface 303 exchanges data and signals with the terminal 200 via the network 100. As an example, the information processing device 300 transmits and receives image data, voice data, screen data, and the like to and from the terminal 200 via the communication interface 303.

The optical drive 307 reads data such as various programs (for example, the program 320) and DBs stored therein from the optical disk 307A or the like, which is an external storage medium, and installs the data in the storage device 310. FIG. 3 illustrates a configuration example in which a necessary program is installed in the information processing device 300 via the optical drive 307, but the present invention is not limited thereto, and the program may be downloaded from another device on the network 100 or the like. In addition, the external storage medium is not limited to the optical disk 307A, and may be, for example, a storage medium such as a USB memory, an SD card, or a CF.

Note that the program 320 may be provided not as a single program but by being incorporated in a part of an arbitrary program. In this case, the processing according to the present embodiment is achieved in cooperation with an arbitrary program. Even a program not including such a part of modules does not depart from the gist of the information processing device 300 according to the present embodiment. In addition, a part or all of the functions provided by the program 320 may be achieved by dedicated hardware.

<C. Software Configuration of Information Processing Device>

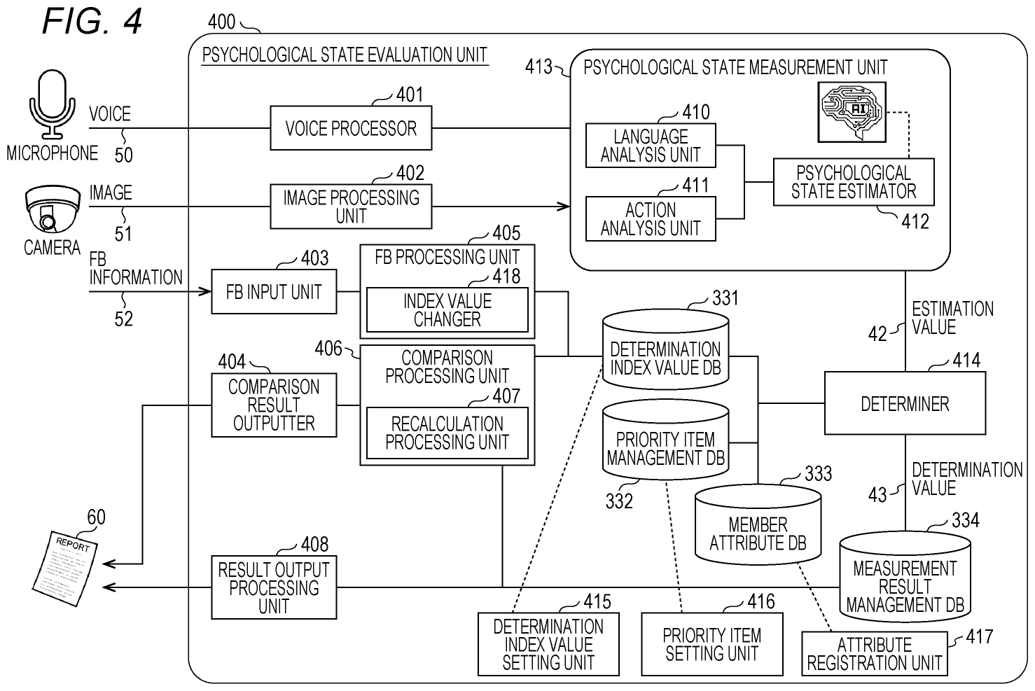

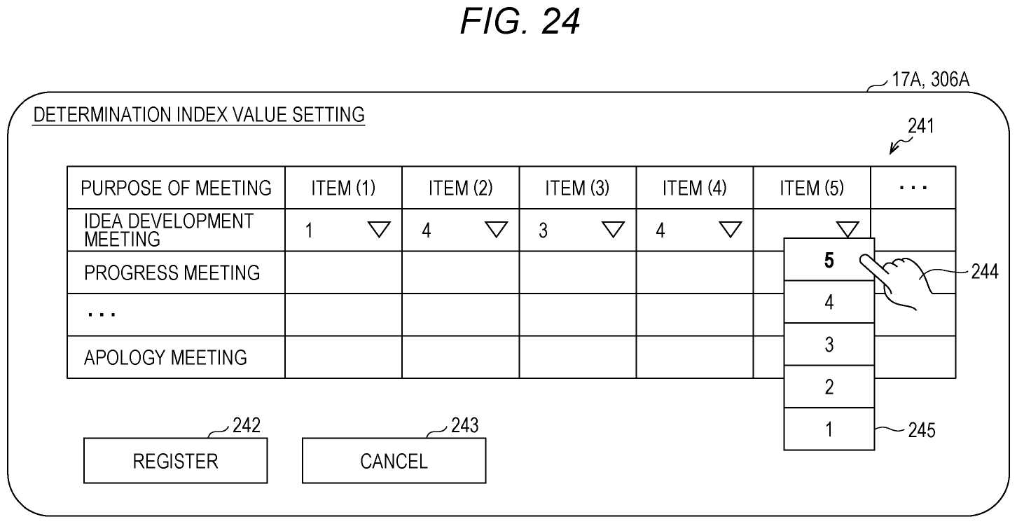

FIG. 4 is a diagram schematically illustrating a software configuration of a psychological state evaluation unit according to the present embodiment. FIGS. 24 to 28 are diagrams illustrating an example of a display screen of the display according to the present embodiment. The psychological state evaluation unit according to the present embodiment includes the program 320 included in the information processing device 300 and software including the DB group 330. The processing performed by the psychological state evaluation unit will be described with reference to the screens of FIGS. 24 to 28 as appropriate.

Referring to FIG. 4, a psychological state evaluation unit 400 includes a psychological state measurement unit 413 that estimates a likelihood that the psychological states of members of a meeting is a predetermined state, a determiner 414 that determines an estimated likelihood according to a purpose of the meeting, and a processing unit that performs information processing related to the determiner 414. The psychological state measurement unit 413 receives voice data 50 of voice collected by the microphone 14 and image data 51 of an image captured by the camera 13 from the terminal 200 while a meeting is being held, and estimates the likelihood that the psychological state of the member is in a predetermined state (state having the psychological safety) from the feature amount regarding the speech of the member included in the received voice data 50 and the image feature amount regarding action of the member included in the image data 51.

The psychological state evaluation unit 400 includes a voice processor 401 and an image processing unit 402 in association with the psychological state measurement unit 413. Furthermore, the processor related to the determiner 414 includes an FB input unit 403 that receives FB (abbreviation of feedback) information 52, an FB processing unit 405 that processes the FB information 52, a result output processing unit 408, a comparison processing unit 406 that performs comparison processing related to an evaluation result, and a comparison result outputter 404 that outputs a comparison result. The psychological state evaluation unit 400 uses various DBs of the DB group 330 for the processing of the determiner 414. More specifically, the DB group 330 includes, for example, a determination index value DB 331, a priority item management DB 332, a member attribute DB 333, and a measurement result management DB 334.

(c1. Configuration of Psychological State Measurement Unit 413)

The psychological state measurement unit 413 estimates the degree (likelihood) of having the psychological safety of the members in the meeting. An example of this estimation method will be described. FIG. 29 is a diagram illustrating a method of psychological state determination according to the present embodiment. The psychological state measurement unit 413 acquires an estimation value for each item from the voice data 50 received from the terminal 200 for each speech while the meeting is being held, and acquires a representative value from the estimation value acquired for each speech for each item. For each item, this representative estimation value is output. Hereinafter, the representative estimation value is referred to as an estimation value 42. The representative value may be an average value, a most frequent value, a median value, or the like of estimation values of a plurality of speeches detected at a meeting.

A method of acquiring the estimation value 42 from the speeches of the members in the meeting will be described. The voice processor 401 detects a voice break in the voice data 50 from the terminal 200, extracts voice data of a speech from the voice data 50 on the basis of the detected break, performs voice recognition processing on the extracted voice data of the speech, and converts the voice data into text data. A language analysis unit 410 of the psychological state measurement unit 413 performs natural language processing on the text data of the speech. Thus, one or more words or clauses 291 constituting a sentence are recognized from the text data. The language analysis unit 410 searches dictionary data, which is not illustrated, on the basis of each word or clause 291 and specifies a type 292. Furthermore, a tone 293 is detected for each word or clause 291. More specifically, the voice data 50 includes data indicating the magnitude of voice in time series from start to end of the speech. The voice processor 401 extracts the magnitude (large, medium, and small) of the voice corresponding to a speech period in which each recognized word or clause 291 is extracted from the voice data 50, as the tone 293 from the time-series data. FIG. 29 illustrates an example of an analysis result of the language analysis unit 41. In FIG. 29, for example, when the speech is “The mistake is a problem. Why did you make such a mistake at this time? Has check been done?”, this speech includes a plurality of words (clauses) 291, word types 292 of respective words (clauses) 291, and the tone 293 as analysis results.

When the speech is analyzed, a psychological state estimator 412 determines an influence value 294 for each word (clause) 291 in FIG. 29 on the basis of the type 292 and the tone 293 according to, for example, the rule base learned for the item (1) “if a mistake is made on this team, it is often criticized.” The influence value 294 is indicated by a value to be deducted from a predetermined score. Since the rule base of the item (1) is learned so that the deduction value of the influence value 294 increases as it is determined to be closer to the content of the speech (words (clauses), tone) that is determined to be “criticized for a mistake,” the estimation value of the item (1) for the speeches decreases.

Here, the rule base of the item (1) has been described as an example, but the psychological state measurement unit 413 stores the learned rule base for the other items (2) to (7) in the storage device 310, for example, and uses the rule base. The psychological state estimator 412 can determine the influence value 294 of each word (clause) of the speech according to the rule base of each of the other items (2) to (7), and output the estimation value for the item from the determined influence value 294.

Thus, the estimation value of each of the items (1) to (7) is acquired for the speech “The mistake is a problem, why did you make such a mistake at this time. Has check been done?”.

In the meeting, a plurality of speeches is actually detected. For each detected speech, the psychological state measurement unit 413 acquires an estimation value of each item of the items (1) to (7), similarly to the procedure described above. At a predetermined timing of the meeting, for example, when the meeting ends, the psychological state measurement unit 413 calculates a representative value (average value, median value, most frequent value, or the like) for each item on the basis of a plurality of estimation values of speeches. The psychological state estimator 412 outputs the representative value of each item calculated in this manner as the estimation value 42.

In the present embodiment, the data used for deriving the estimation value 42 is not limited to the voice data 50, and is only required to be data that is observed for the members during the meeting. For example, the image data 51 may be added as the observed data. More specifically, the image processing unit 402 extracts a partial image of the member from each frame image of the image data 51. An action analysis unit 411 analyzes the extracted partial image of the member, and extracts an image feature amount regarding the action of the person. The feature amount (the type 292 of the word (clause) and the tone 293) based on the speech of the member and the image feature amount regarding the action may be input to the rule base as the meeting information that is information obtained by observing the member in the meeting, and the rule base as the learned estimation model may derive the likelihood that the member has the psychological safety as the estimation value 42 of the estimation result. The image feature amount of the action may include an image feature amount representing a person's action reflecting a psychological state, for example, eye movement, hand movement, and the like.

Note that the psychological state measurement unit 413 includes a rule base learned as a learning model for deriving the estimation value 42, but the learning model for deriving the heart estimation value 42 is not limited to the learned rule base.

(c2-1. Configuration Using Determination Index Value DB 331 of Determiner 414)

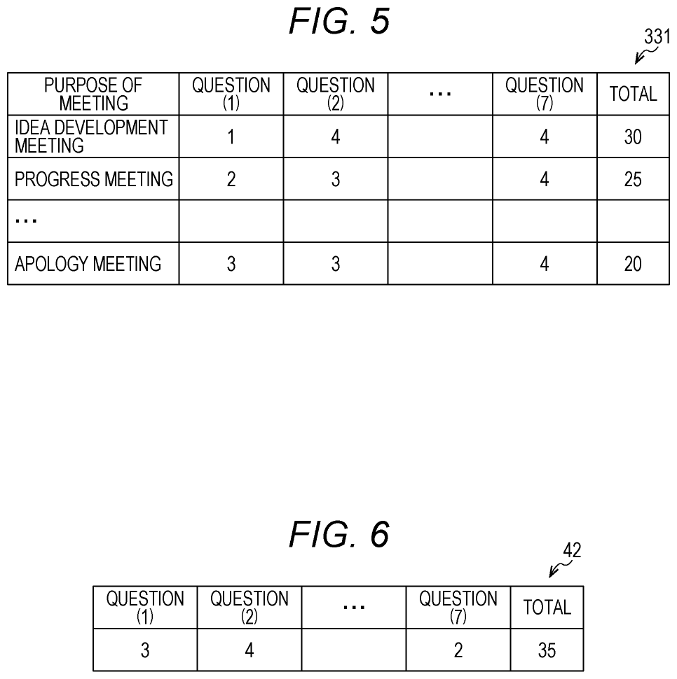

The determiner 414 determines the likelihood indicated by the estimation value 42 of each item output from the psychological state measurement unit 413 on the basis of the index value corresponding to the item in the determination index value DB 331, and outputs a determination value 43 indicating the result. FIG. 5 is a diagram schematically illustrating an example of the determination index value DB 331 according to the present embodiment. Referring to FIG. 5, in the determination index value DB 331, as the index value for each item (each of the questions (1) to (7) described above), a default value or the like can be set in advance by performing the above-described experiment or the like for each purpose of the meeting (for example, an idea development meeting, a progress meeting, an apology meeting, and the like). The index value is a value used to determine the likelihood (magnitude of likelihood) that the members in the meeting have the psychological safety from the estimation value 42 of each item estimated for the meeting.

FIG. 24 illustrates an example of a user interface (UI) screen for setting an index value in the determination index value DB 331. A determination index value setting unit 415 causes a UI screen of FIG. 24 to be displayed on the display 306A of the information processing device 300 or the display 17A of the terminal 200. In a case where the UI screen is displayed on the display 17A, the determination index value setting unit 415 transmits a web page to the terminal 200, and the web browser of the terminal 200 displays a web screen based on the web page from the determination index value setting unit 415 on the display 17A.

On the UI screen of FIG. 24, the user can designate and set the index value 241 for each item for each purpose of the meeting by operating a cursor 244 from a pull-down menu 245. When a registration button 242 is operated, the determination index value setting unit 415 registers the set index value of each item in the determination index value DB 331. The user can cancel the registration operation by operating a cancel button 243.

FIG. 6 is a diagram illustrating an example of the estimation value 42 according to the present embodiment. The estimation value 42 in FIG. 6 indicates, for example, an estimation value for each item estimated for the progress meeting.

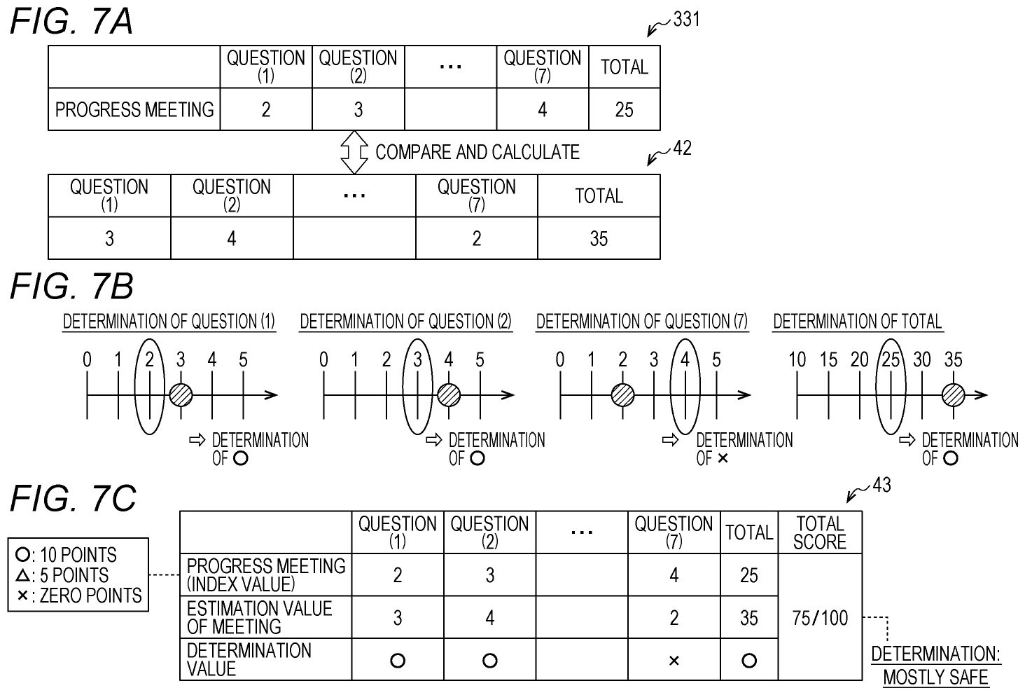

FIGS. 7A to 7C are diagrams illustrating a determination method of the determiner 414 according to the present embodiment. Referring to FIGS. 7A to 7C, the determiner 414 retrieves the index value of each item corresponding to the progress meeting in FIG. 7A from the determination index value DB 331 on the basis of the purpose of the meeting, that is, the progress meeting. The determiner 414 compares the estimation value 42 of each item with the index value corresponding to the item, and outputs the determination value 43 for the item on the basis of the comparison result.

For example, upon determining that the comparison result satisfies a condition (estimation value 42≥index value) for each item, the determiner 414 outputs “Good (indicated by ◯ in the diagram)” (10 points) indicating that the estimation value 42 of the item has a high likelihood of the psychological safety, but upon determining that the condition is not satisfied, the determiner 414 outputs “NG (indicated by x in the diagram)” (0 points) indicating that the estimation value 42 has a low likelihood of the psychological safety. In addition, in a case where the comparison result satisfies a condition (estimation value 42<(index value−1)), the determiner 414 may output 5 points in the middle.

The determiner 414 sums up the output values (any of 10 points, 5 points, and 0 points) of the respective items, compares the total value with the total value of the determination index value DB 331, and upon detecting that the result of the comparison satisfies a condition (total value of output values of respective items≥total value of indexes), the determiner 414 outputs “Good” qualitatively indicating that the members of the meeting are highly likely to have the psychological safety as the determination value 43, as illustrated in FIG. 7B. As described above, the determination value (the magnitude of the likelihood of having the psychological safety) determined for each of the plurality of items is converted into a numerical value, but the value to be converted is not limited to 10 points, 5 points, and 0 points, and the like.

The determiner 414 sums up the output values of the respective items, compares the total value with the total value of the determination index value DB 331, and upon detecting that a result of the comparison satisfies a condition (total value of output values of respective items<total value of indexes), the determiner 414 outputs “NG” as a qualitative value indicating that the likelihood that the members of the meeting have the psychological safety is low as the determination value 43.

In addition, the determiner 414 may indicate the determination value 43 as a quantitative value. For example, in a case where the total value of the indexes is on a scale of 100 points, how many points the total value of the output values of the respective items occupies on a scale of 100 points may be calculated, and the likelihood of having the psychological safety may be determined to be high when the calculation result exceeds a threshold value, or a ratio of the total value of the output values of the respective items to the total value of the indexes may be calculated, and the calculated value may be output as the determination value 43. For example, in FIG. 7C, this ratio is calculated as “75”, and the determiner 414 detects that “75” exceeds the threshold, and consequently determines that the likelihood of having the psychological safety is high (“Good”). In addition, the determiner 414 may output both or one of a qualitative value of “Good” or “NG” and a calculated quantitative value (such as “75”) as the determination value 43.

(c2-2. Configuration Using Priority Item Management DB 332 of Determiner 414)

Depending on the purpose of the meeting, there are items that are particularly given priority in evaluating whether there is psychological safety. The determiner 414 compares the estimation value 42 of the priority item with the corresponding index value of the determination index value DB 331, and when it is detected that the comparison result does not satisfy a condition (estimation value≥index value), even if the estimation values 42 of other items satisfy the condition, the determination value 43 is output as “NG”.

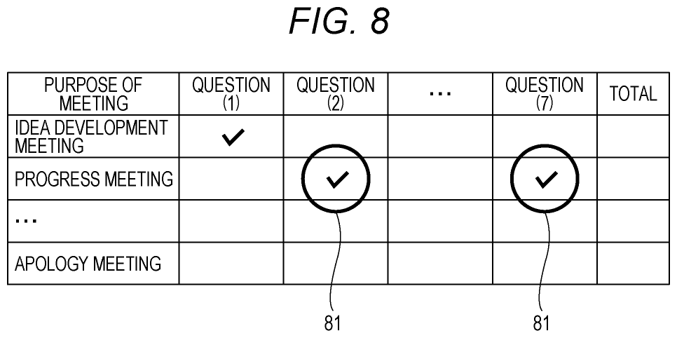

For each purpose of the meeting, items with priority are managed by the priority item management DB 332. FIG. 8 is a diagram schematically illustrating an example of the priority item management DB 332 according to the present embodiment. Referring to FIG. 8, the priority item management DB 332 has priority items set in advance for each purpose of the meeting. In FIG. 8, for the progress meeting, for example, the item (2) and the item (7) are set as priority items 81.

The item (2) indicates that “members of this meeting are able to bring up problems and negative issues.” In the progress meeting, the item (2) can be said to be an important item in determining the likelihood that the members have the psychological safety such that they are likely to actively speak about delay in progress.

Furthermore, the item (7) indicates that “when advancing work with current meeting members, it feels that my skills are exhibited.” In the progress meeting, the item (7) can be said to be an important item in determining the likelihood of having the psychological safety such that members share the progress of work and bring positive thinking to the members in advancing work.

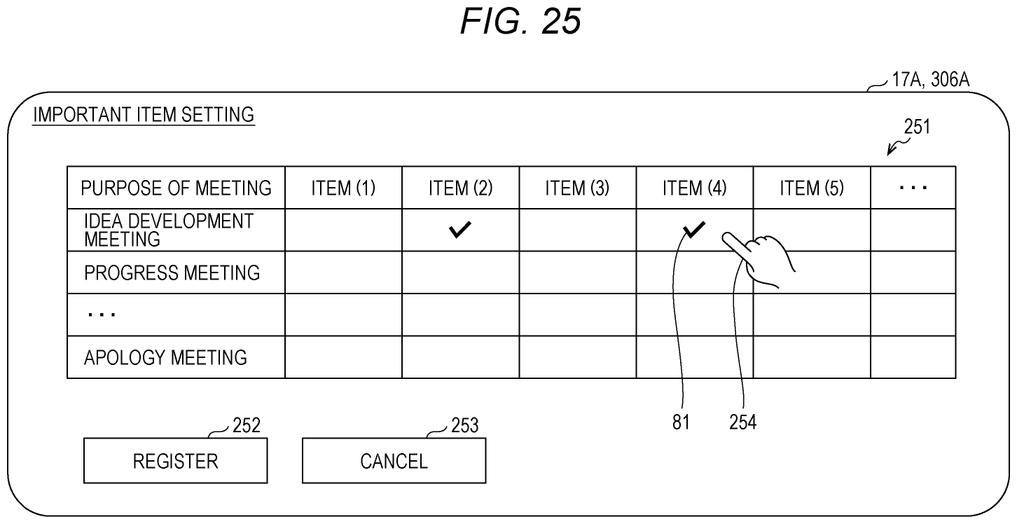

FIG. 25 illustrates an example of a UI screen for setting a priority item in the priority item management DB 332. A priority item setting unit 416 causes the UI screen of FIG. 25 to be displayed on the display 306A of the information processing device 300 or the display 17A of the terminal 200. In a case where the UI screen is displayed on the display 17A, the priority item setting unit 416 transmits a web page to the terminal 200, and the web browser of the terminal 200 displays a web screen based on the web page from the focused item setting unit 416 on the display 17A.

On the UI screen of FIG. 25, the user can set a desired item as a priority item 81 by operating the cursor 244 to designate the desired item for each purpose of the meeting. When the registration button 252 is operated, the priority item setting unit 416 registers the priority item 81 in the priority item management DB 332. The user can cancel the registration operation by operating a cancel button 253.

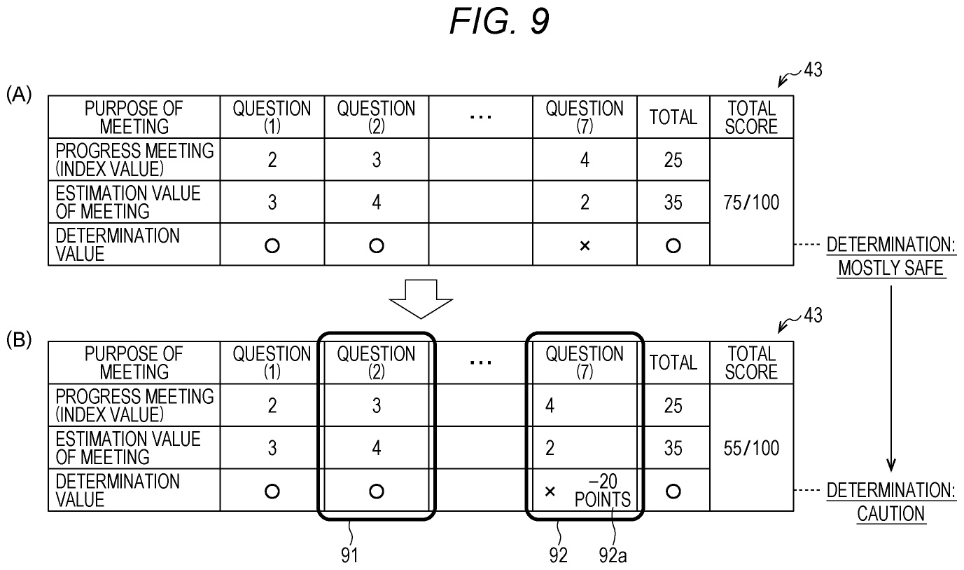

FIG. 9 is a diagram illustrating an example of a determination result using the priority item management DB 332 of the determiner 414 according to the present embodiment. (A) of FIG. 9 illustrates the determination value 43 obtained by the determiner 414 determining the estimation value 42 of the progress meeting by using only the index value of the determination index value DB 331. In contrast, (B) of FIG. 9 illustrates the determination value 43 obtained by the determiner 414 determining the same estimation value 42 on the basis of the determination index value DB 331 and the priority item 81 of the priority item management DB 332. In (A) of FIG. 9, the determination value 43 indicates “Good” and 75 points, and it is determined that the member is highly likely to have the psychological safety in the progress meeting. However, in (B) of FIG. 9, the determination value 43 indicates “NG” and 55 points, and it is determined that the member is less likely to have the psychological safety in the progress meeting and it is necessary to be cautious. This difference is because the determination value 43 has been adjusted. Specifically, this is because it has been detected 91 that the condition (estimation value≥index value) is satisfied for the estimation value 42 of the item (2) set in the priority item 81 but it has been detected 92 that the condition (estimation value≥index value) is not satisfied for the estimation value 42 of the item (7), and a −20 adjustment value 92a is added to the determination value 43.

As illustrated in FIG. 9, when the above condition is not satisfied for the priority item 81, the negative adjustment value 92a is added to the determination value 43, and as a result, the determination becomes (75−20)=55 points. When the above condition is satisfied for the priority item 81, the adjustment value 92a that is negative with respect to the determination value 43 is not performed. Note that the adjustment value 92a for the priority item 81 is not limited to −20 points, and can be set by the user. For example, the adjustment value 92a may be set to a negative or positive value. In this manner, the determination value 43 of the priority item can be adjusted by the adjustment value 92a.

(c2-3. Output of Result)

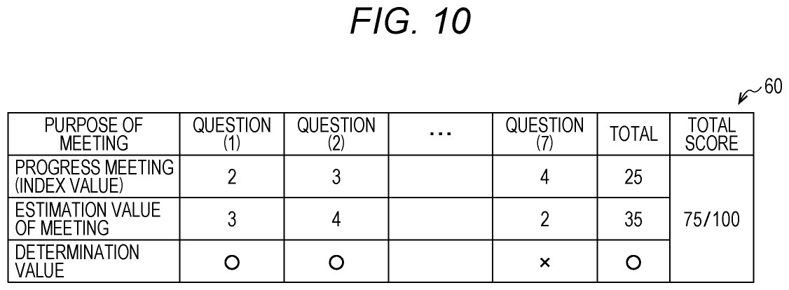

FIG. 10 is a diagram schematically illustrating an example of a report 60 according to the present embodiment. The determination value 43 for the meeting output by the determiner 414 is stored in the measurement result management DB 334. The result output processing unit 408 retrieves the determination value 43 of the meeting from the measurement result management DB 334 and outputs the retrieved determination value 43. The result output processing unit 408 displays the determination value 43 on the display 306A of the information processing device 300, for example. Alternatively, the result output processing unit 408 may transfer the determination value 43 to the terminal 200 so as to be displayed on the display 17A. Note that the output device of the determination value 43 is not limited to the display, and may be a printer or the like.

The determination value 43 is output in the form of the report 60 illustrated in FIG. 10, for example. Referring to FIG. 10, the report 60 includes the purpose of the meeting, the index values of the items (1) to (7), the estimation value 42, the determination value (Good or NG), and a score (75/100 points in FIG. 10) obtained by quantitatively determining how much psychological safety is present.

The report 60 may include an execution date of the meeting, the purpose of the meeting, the number of members of the meeting and identification information, and an index value (if it is a default value, an indication that it is a default index value) used to derive the determination value 43.

(c3. Change of Index Value)

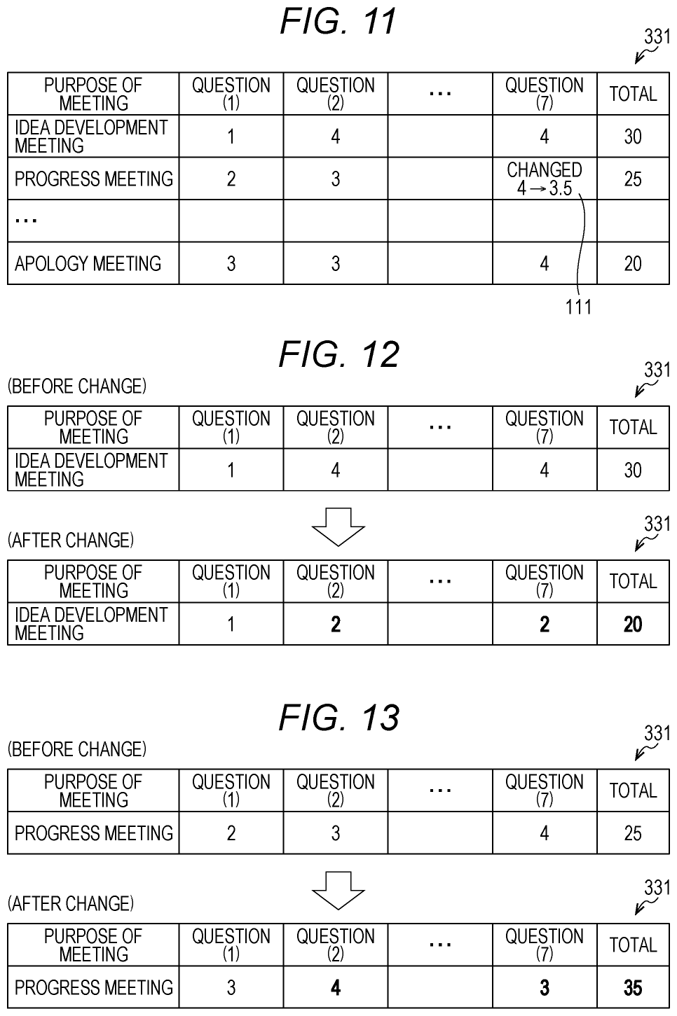

In the present embodiment, the user can set and change the index value of the determination index value DB 331. When the user has performed a setting operation for an index value on the input device 16A or 305A, the determination index value setting unit 415 sets the index value in the determination index value DB 331 on the basis of the user operation received by the input device 16A or 305A via the input interface. Note that users permitted to set or change the index value may include a meeting member. FIG. 11 is a diagram illustrating another configuration example of the determination index value DB 331 according to the present embodiment.

For example, the members of the progress meeting self-evaluate the report 60, and input, to the psychological state evaluation unit 400, feedback (abbreviated as “FB” in FIG. 4) information 52 that reflects a result of the self-evaluation on the determination index value DB 331.

For example, when a member self-evaluates that the “evaluation of the item (7) is “NG” at all times” contrary to the high psychological safety felt in the progress meeting, the member determines that “the index value of the item (7) may be high”. In this case, the member inputs the FB information 52 for changing the index value of the item (7) of the progress meeting. The FB input unit 403 receives the FB information 52 from the user, and outputs this information to the FB processing unit 405. The FB processing unit 405 extracts the purpose of the meeting for which the index value is to be changed, the identifier of the item to be changed, and the index value after change from the FB information 52 of the user, and outputs an extraction result to an index value changer 418.

On the basis of the extraction result, the index value changer 418 retrieves the item to be changed of the meeting to be changed from the determination index value DB 331, and sets the index value after change indicated by the extraction result to a retrieved item. Thus, in the determination index value DB 331, for example, the index value of the item (7) corresponding to the meeting having the purpose of the progress meeting is changed to a new index value designated by the member in the FB information 52. Referring to FIG. 11, a change 111 from 4 to 3.5 is made on the index value of the item (7) corresponding to the meeting of the progress meeting.

Note that the FB processing unit 405 may perform the above-described change the index value as follows. For example, when the FB information 52 for the same item of a meeting for the same purpose is received from a predetermined number of members, the FB processing unit 405 may calculate a representative value (average value, most frequent value, median value, or the like) from the index value indicated by the FB information 52 received from each member and output the representative value to the index value changer 418 as the index value after change.

As described above, the index value changer changes the index value on the basis of a user operation received by the input interface, or changes the index value on the basis of a user operation when the user operation is received from a plurality of users.

Furthermore, the change of the index value by the FB processing unit 405 is not limited to the changing method using the FB information 52. For example, when it is detected that there is a difference between the index value of a certain item of a meeting of a certain purpose and the index value of a corresponding item of a meeting of another purpose (for example, when it is detected that the difference exceeds a threshold), the FB processing unit 405 calculates a median value based on the difference. Then, the index value changer 418 changes (corrects) the index value of the certain item so as to indicate the median value. This median value is an intermediate value between the index value of the certain item of the meeting of the certain purpose and the index value of the corresponding item of the meeting of another purpose.

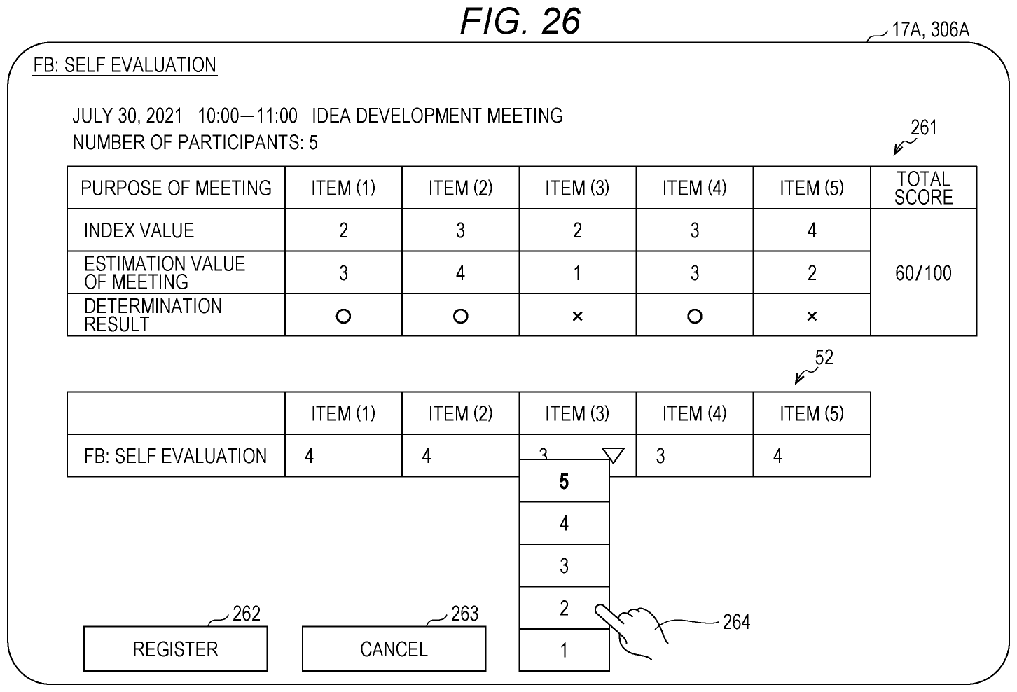

FIG. 26 illustrates an example of a UI screen for receiving the FB information 52. The FB input unit 403 causes the UI screen of FIG. 26 to be displayed on the display 306A of the information processing device 300 or the display 17A of the terminal 200. In a case where the UI screen is displayed on the display 17A, the FB input unit 403 transmits a web page to the terminal 200, and the web browser of the terminal 200 displays a web screen based on the web page from the FB input unit 403 on the display 17A.

On the UI screen of FIG. 26, for example, the report 60 of an idea development meeting and the FB information 52 are displayed. The user can self-evaluate the report 60 and designate the index value by operating the cursor 264 from the pull-down menu for a desired item, and can thereby set the designated index value to the desired item When the registration button 262 is operated, the FB input unit 403 inputs the FB information 52 set on the UI screen of FIG. 26. The user can cancel the input of the FB information 52 by operating a cancel button 263.

(c4. Comparison of Determination Values Before and After Change of Index Value)

In the present embodiment, the comparison processing unit 406 retrieves the determination value of each item of the meeting from the measurement result management DB 334 for each purpose of the meeting, edits a retrieved determination value into comparison result data 271 having a format that can be compared in order of the date when the meeting was held, and outputs the comparison result data 271 to the comparison result outputter 404. The comparison result outputter 404 causes the information processing device 300 or the terminal 200 to output the comparison result data 271 from the comparison processing unit 406. Thus, it is possible to output the determination value 43 determined by the determiner 414 for each of a plurality of meetings held at different times and having the same purpose in which the members participate.

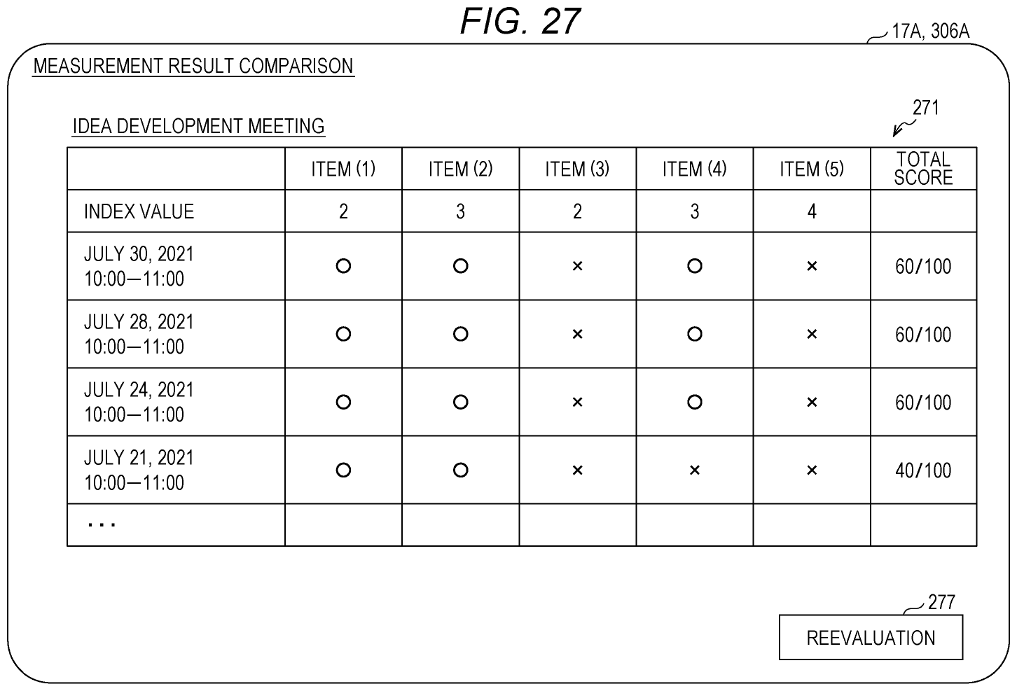

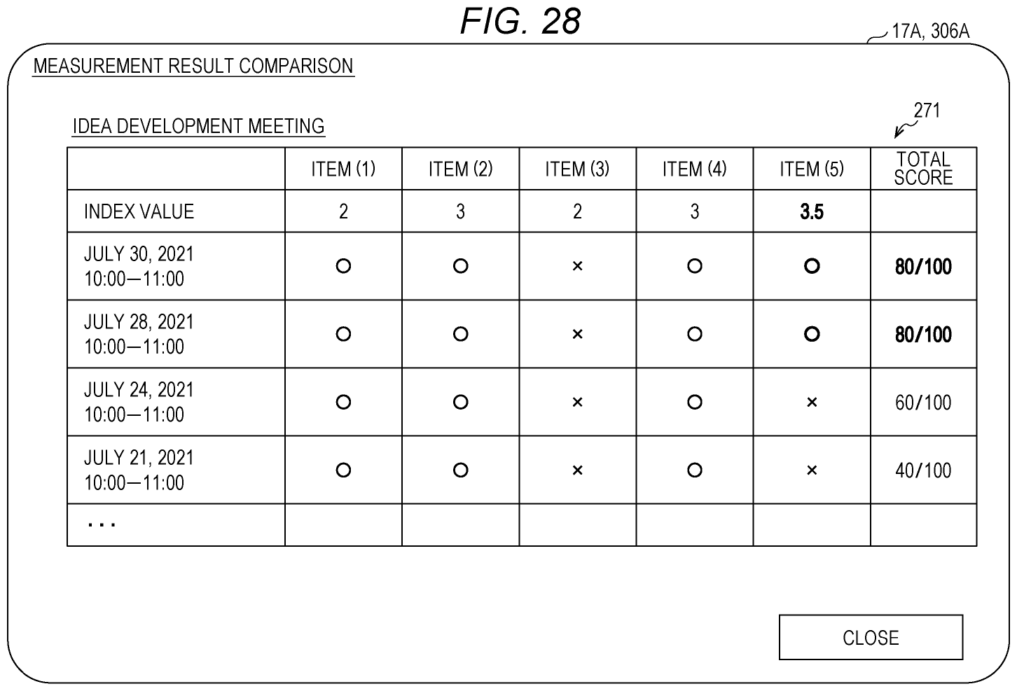

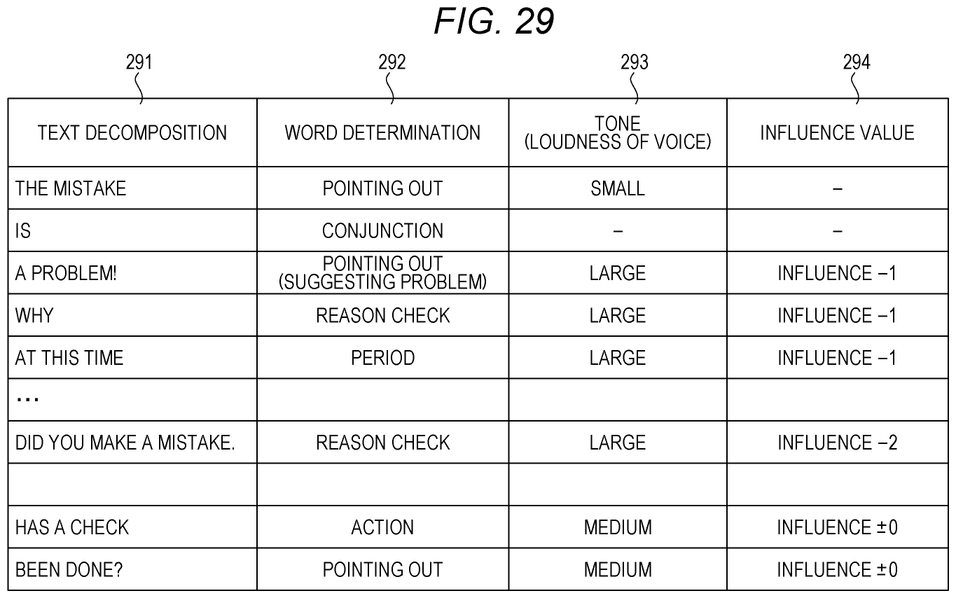

FIGS. 27 and 28 illustrate examples of a UI screen for displaying the comparison result data 271. The UI screen of FIG. 27 is displayed on the display 306A of the information processing device 300 or the display 17A of the terminal 200 by the comparison result outputter 404. In a case where the UI screen is displayed on the display 17A, the comparison result outputter 404 transmits a web page to the terminal 200, and the web browser of the terminal 200 displays a web screen based on the web page from the comparison result outputter 404 on the display 17A.

On the UI screen of FIG. 27, for example, for each of four idea development meetings held from July 21 to Jul. 30, 2021, the determination values of respective items of the meeting are displayed in a list form in association with the index values of the items in order of the date. From the list in FIG. 27, the user can confirm how much degree the psychological safety of the members has changed over a repetition of meetings by holding the idea development meetings multiple times, or can confirm when a meeting with a high or low degree of the psychological safety is held.

In a case of displaying the UI screen of the comparison result data 271 based on the index value changed by the index value changer 418, the user operates the button 277 to instruct “reevaluation” based on the index value after change. Upon receiving the user operation of the button 277, the comparison result outputter 404 outputs the received user operation to the comparison processing unit 406. The comparison processing unit 406 outputs a reevaluation instruction to a recalculation processing unit 407 on the basis of the user operation.

Upon receiving the reevaluation instruction, the recalculation processing unit 407 recalculates the determination value of each item of the meeting for each of the above-described four idea development meetings on the basis of the index value after change that has been changed by the determination index value setting unit 415. More specifically, the recalculation processing unit 407 retrieves the estimation value 42 of each item from the measurement result management DB 334 for each of the above-described four idea development meetings, compares the retrieved estimation value 42 of each item with the index value after change, and calculates the determination value (Good, NG, and total score) on the basis of whether or not a comparison result satisfies a condition (estimation value 42≥index value). The comparison result outputter 404 displays the comparison result data 271 including the determination values recalculated using the index values after change for the above-described four idea development meetings. An example of a display is illustrated on the UI screen of FIG. 28.

On the UI screen of FIG. 28, for each of the four idea development meetings, the determination values of respective items of the meeting are displayed in a list form in association with the index values used for the calculation in order of the date. On the UI screen of FIG. 28, the index value of the item (5) has been changed from the original value of 4.0 to a lower value of 3.5. The determination value of the item (5) in FIG. 28 recalculated according to this change is larger (improved) in the degree of the psychological safety than the determination value of the item (5) in FIG. 27.

When the user confirms the improvement in the determination value from the UI screen in FIG. 28, even if the degree of having the psychological safety in FIG. 27 is low, the user can confirm that the cause is not the member but the index value used for the evaluation. As described above, the psychological state evaluation unit 400 can provide the comparison result data 271 before and after the change of the index value in FIGS. 27 and 28 to the user as the support information for searching for the cause of impairment of the psychological safety of the members in the meeting.

(c5. Change Index Value According to Attribute of Meeting Member)

In the present embodiment, the index value changer 418 changes (corrects) the index value of the determination index value DB 331 in response to determining that the member indicates a predetermined attribute or there is a predetermined number of members having a predetermined attribute on the basis of the attribute of the member of the meeting retrieved from the member attribute DB 333 in which the attribute of the member is registered. FIGS. 12 and 13 are diagrams illustrating examples of indexes before and after the change in association with each other. FIG. 12 illustrates a case where the index value changer 418 changes the index values of the item (2) and the item (7) among the index values of, for example, the idea development meeting in the determination index value DB 331 according to the attribute of the member of the idea development meeting retrieved from the member attribute DB 333. FIG. 13 illustrates a case where the index value changer 418 changes the index values of the item (1), the item (2), and the item (7) among the index values of, for example, the progress meeting in the determination index value DB 331 according to the attribute of the member of the progress meeting retrieved from the member attribute DB 333.

For example, in the idea development meeting of an organization such as a company, a constituent of the organization participates in the meeting as a member. It is known that the degree of the psychological safety of a member varies depending on attributes of other members, for example, a position in an organization or the like. For example, although the idea development meeting originally has an atmosphere in which members can freely speak, this atmosphere is impaired when a predetermined number of members with high positions in an organization such as a manager or a director is included in other members. Accordingly, it can be assumed that innocuous speeches from the members increases. Therefore, the index value of the item (2) “members of this meeting are able to bring up problems and negative issues” illustrated in FIG. 12 is changed to a low value. Furthermore, since it is assumed that another member (for example, a manager, a director, or the like) makes a negative speech or a coercive speech, the index value of the item (7) “when advancing work in the current meeting, it feels that my skills are exhibited” is changed to a low value.

Furthermore, for example, in a case where the members of the progress meeting of a project includes a rookie, other members give an instructive speech to or warn the rookie in order to efficiently proceed with the project. That is, in this progress meeting, since it is assumed that the number of instructional speeches or caution speeches increases, the index value of the item (2) “members of this team are able to bring up problems and negative issues” illustrated in FIG. 13 is changed to a high value. In addition, the index value of the item (7) “when advancing work in the current meeting, it feels that my skills are exhibited” is changed to a low value so that it is possible to reliably evaluate whether the instruction speech or the caution speech is coercive for the rookie, that is, the degree of the psychological safety of the rookie is not lowered.

In the present embodiment, an attribute registration unit 417 acquires the attribute of a member of the meeting on the basis of a user operation of the input device 16A or 305A via the input interface, and registers the acquired attribute of each member in the member attribute DB 333. In a case where the estimation value 42 of the meeting is evaluated, the index value changer 418 specifies an item to be changed among respective items of the meeting of the determination index value DB 331 according to the attribute of each member retrieved from the member attribute DB 333 and a predetermined rule based on the purpose of the meeting as described above, and changes the index value of the specified item to a predetermined index value. The determiner 414 determines the estimation value 42 of each item of the meeting on the basis of the index value after change. Thus, it is possible to determine the degree of the psychological safety of the member in the meeting according to the attribute of the member. Note that the above-described rule used by the index value changer 418 is acquired in advance by experiment or the like.

<D. Flowchart>

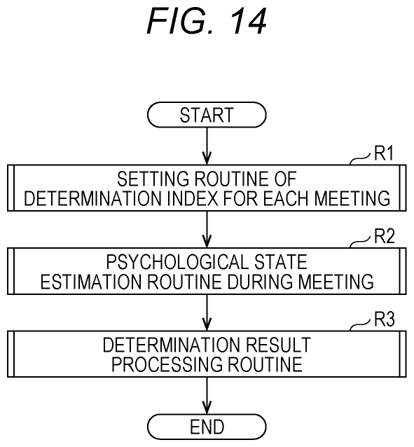

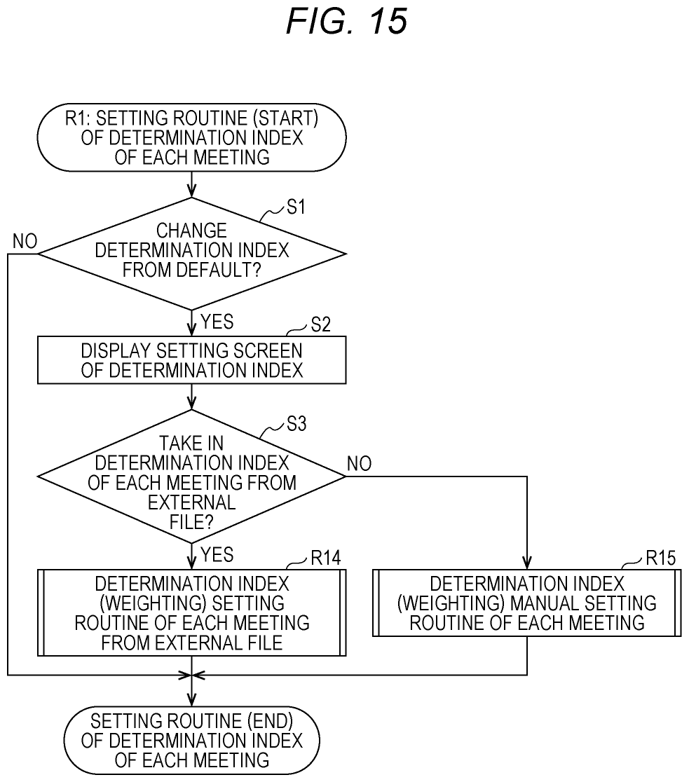

Processing according to the present embodiment will be described with reference to flowcharts of FIGS. 14 to 23. Processing of these flowcharts is achieved by the processor 301 executing the program 320. FIG. 14 is a schematic flowchart of overall processing according to the present embodiment. Referring to FIG. 14, the processor 301 performs routines R1, R2, and R3. In the routine R1, the processor 301 sets an index value for each item in the determination index value DB 331 for each meeting, that is, for each purpose of the meeting as the determination index value setting unit 415. In the routine R2, the processor 301 measures the degree of the psychological safety of the members with respect to the meeting as the psychological state measurement unit 413, and outputs the measurement result as the estimation value 42 of each item. In the routine R3, the processor 301 determines the estimation value 42 measured and output for the meeting as the determiner 414, and outputs the determination value 43.

(d1. Setting Routine of Determination Index Value of Each Meeting: R1)

FIG. 15 is a flowchart of the routine R1 of FIG. 14. Referring to FIG. 15, the processor 301 determines whether or not to change the index value of the meeting from the default value (for example, an initial value) on the basis of the user operation (step S1), and when it is determined not to change the index value (NO in step S1), the processing is ended, or when it is determined to change the index value (YES in step S1), the screen of FIG. 24 for setting the index value is displayed on the display (step S2). The processor 301 determines whether or not to take in (read) the determination index value of the meeting from the external file on the basis of the user operation (step S3).

When it is determined to take in the index value from the external file (YES in step S3), the processor 301 executes the routine R14 for automatically setting the determination index value from the external file to the meeting, or when it is determined not to take in the index value from the external file (NO in step S3), the processor 301 executes the routine R15 for setting the manually set index value as the determination index value of the meeting.

(d1-1. Setting Routine of Determination Index Value of Each Meeting by External File: R14)

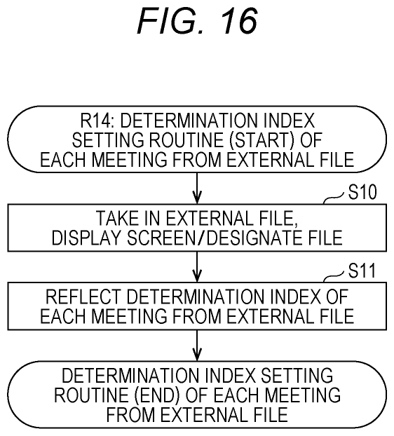

FIG. 16 is a flowchart of the routine R14 of FIG. 15. Referring to FIG. 16, the processor 301 causes a list of possible external files to be displayed on the display, receives an operation to designate a desired file from the user (step S10), reads the index value of each item of each meeting from the designated external file, and reflects (sets) the index value as the index value of each item of the meeting in the determination index value DB 331 (step S11).

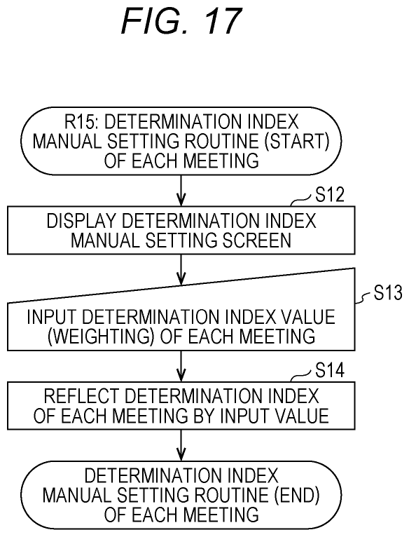

FIG. 17 is a flowchart of the routine R15 of FIG. 15. Referring to FIG. 17, the processor 301 causes the UI screen of FIG. 24 to be displayed on the display in order to manually set the index value (step S12), inputs the index value of each item of each meeting based on the user operation received via the UI screen (step S13), and reflects (sets) the input index value of each item of each meeting as the index value of each item of the meeting in the determination index value DB 331 (step S14).

The external file includes, for example, a file storing an index value of the determination index value DB 331 of a meeting of another project team. More specifically, in a case where a user of a certain department or team wants to change the index value applied to a meeting for a certain purpose, a file having the index value applied to each item of a meeting for the same purpose of another department or another team can be used as the external file.

In addition, when changing the index value, the index value changer 418 may change the original index value according to a change rule. The change rule includes, for example, a rule of change based on index values for a plurality of meetings having the same purpose. In addition, the change rule may include a rule for changing the index value of the target meeting on the basis of the index values for a plurality of meetings having the same purpose in which constituent members are different. For example, in a case where a difference between the original index value and the new index value exceeds a threshold, the rule of rewriting (changing) the original index value to an intermediate value between the two may be included. The change rule may be a combination of these rules, and the rule is not limited thereto.

(d2-1. Psychological State Estimation Routine During Meeting: R2)

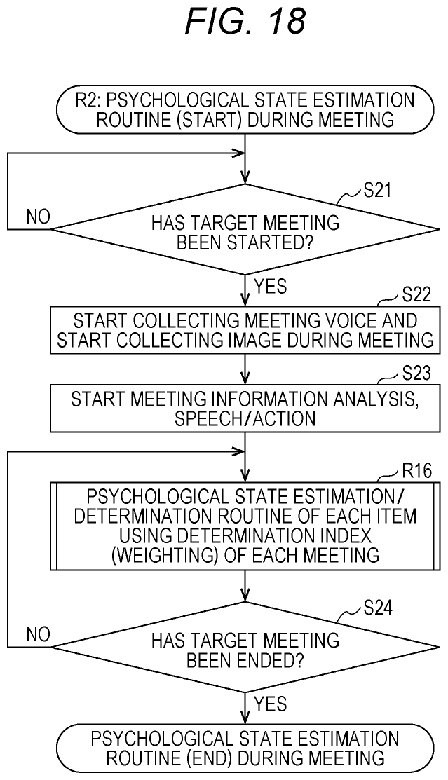

FIG. 18 is a flowchart of the routine R2 of FIG. 14. Referring to FIG. 18, the processor 301 retrieves meeting schedule data, which is not illustrated, on the basis of the current time, and determines whether or not the meeting to be estimated has been started on the basis of a retrieval result (step S21). When it is determined that the target meeting has not been started (NO in step S21), the processor 301 repeats the processing in step S21, or when it is determined that the target meeting has been started (YES in step S21), the processor 301 detects and starts collecting text data of a speech from the voice data 50 transferred and received from the terminal 200 as the voice processor 401 and detects and starts collecting an image feature amount indicating an action of a member from the image data 51 transferred and received from the terminal 200 as the image processing unit 402 (step S22).

The processor 301 analyzes meeting information which is information observed from members in a meeting including the text data of speeches from the voice processor 401 and image feature amounts of actions from the image processing unit 402 (step S23). More specifically, the processor 301 analyzes the text data of the speech as the language analysis unit 410, and outputs, for example, the type 292 and the tone 293 corresponding to each word (clause) 291 illustrated in FIG. 29 as data of an analysis result. In addition, the processor 301 outputs, as the image processing unit 402, a feature amount representing an action (for example, eye movement and hand movement) of the member of the image data 51 as meeting information which is information observed from the member.

The processor 301 determines whether or not the target meeting has ended while performing the routine R16 of the psychological state estimation and evaluation in the meeting (step S24). When it is not determined that the target meeting has ended (NO in step S24), the routine R16 is repeated, or when it is determined that the target meeting has ended (YES in step S24), the process ends. Note that the end of the meeting is determined on the basis of whether or not a speech indicating the end of the meeting is detected by voice recognition processing based on the voice data 50, or whether or not the member performs a logout operation.

(d2-2. Psychological State Estimation and Evaluation Routine: R16)

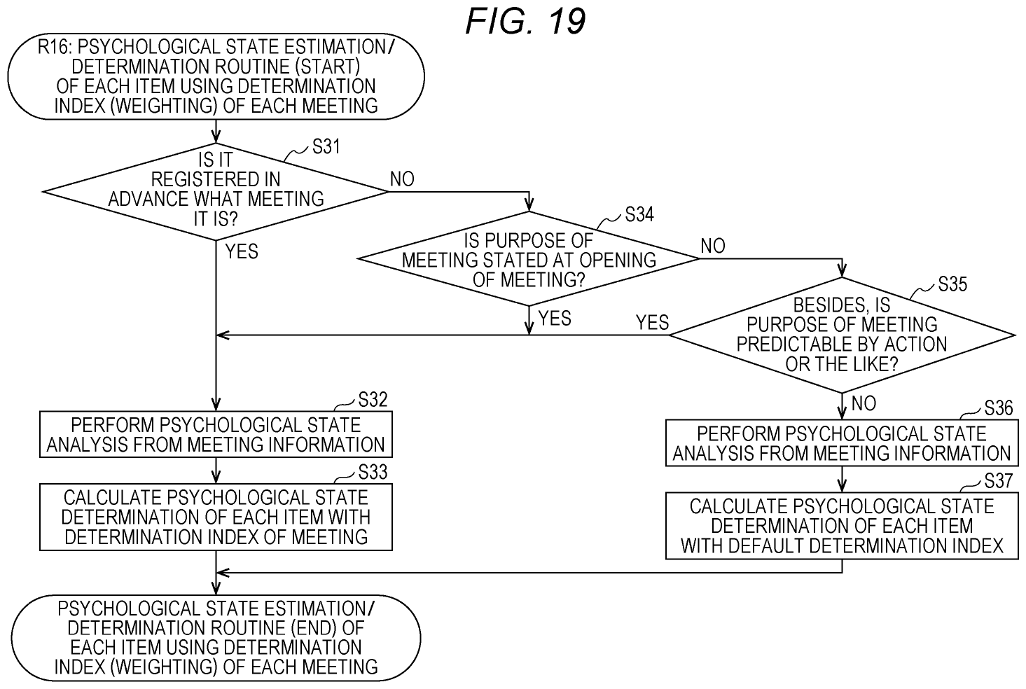

FIG. 19 is a flowchart of the routine R16 of FIG. 18. Referring to FIG. 19, the processor 301 retrieves, for example, meeting schedule data that is information registered regarding the meeting (purpose, member identification information, and the like), and determines whether the purpose of the target meeting is registered on the basis of a retrieval result (step S31). When it is determined that the purpose has been registered (YES in step S31), the processor 301 proceeds to step S32, or when it is determined that the purpose has not been registered (NO in step S31), the processor 301 determines whether the purpose of the meeting has been spoken at the opening of the meeting on the basis of speech data derived by the voice recognition processing of the voice data 50 (step S34). When it is determined that the purpose of the meeting has been spoken (YES in step S34), the process proceeds to step S32. For example, the purpose of the meeting is acquired by performing recognition processing on voices collected during the meeting (for example, at the beginning). Furthermore, when it is determined that the purpose of the meeting has not been spoken (NO in step S34), it is determined whether the purpose of the meeting can be specified from action data (step S35). When the purpose of the meeting can be specified (YES in step S35), the process proceeds to step S32. When the purpose of the meeting cannot be specified (NO in step S35), the processor 301 outputs, as the psychological state measurement unit 413, the estimation value 42 of the psychological state for each item of the target meeting on the basis of the analysis result of the meeting information, and outputs, as the determiner 414, the determination value 43 using the default index value (step S37), and the process ends.

In a case where the purpose of the meeting has been determined, the processor 301 outputs the estimation value 42 of each item of the target meeting on the basis of the meeting information as the psychological state measurement unit 413 (step S32). The processor 301 compares, as the determiner 414, the estimation value 42 of each item output in step S32 with the index value of each item corresponding to the meeting of the purpose retrieved from the determination index value DB 331, and calculates the determination value 43 (Good or NG) of each item on the basis of whether or not the comparison result satisfies the above condition (step S33). The estimation value 42 and the determination value 43 of each item of the target meeting are stored in the measurement result management DB 334. Thereafter, the process ends.

(d3-1. Determination Result Processing Routine: R3)

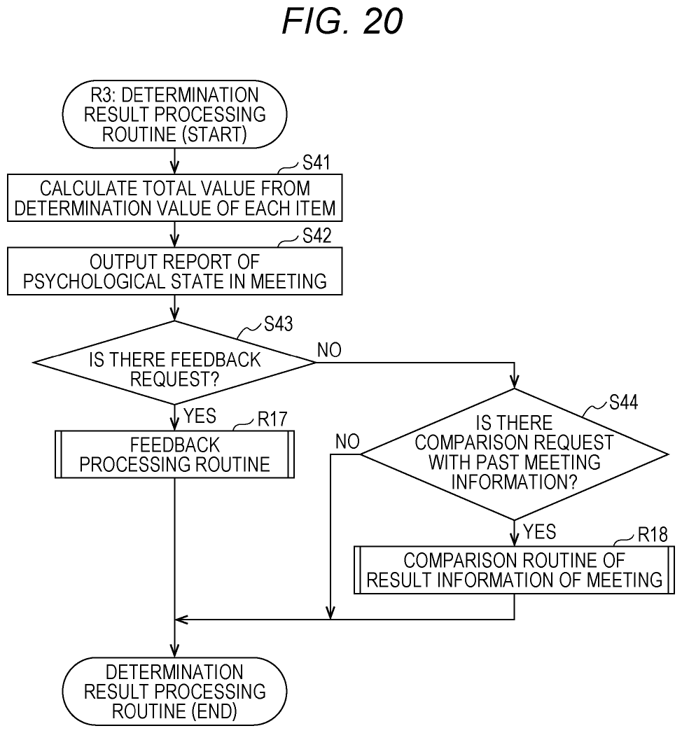

FIG. 20 is a flowchart of the routine R3 of FIG. 14. Referring to FIG. 20, the processor 301 calculates a score based on the determination value 43 of each item of the target meeting as the determiner 414, and stores the score in the measurement result management DB 334 (step S41).

The processor 301 retrieves the estimation value 42, the determination value 43, and the score of each item of the target meeting from the measurement result management DB 334 as the result output processing unit 408, and causes information based on retrieval results to be displayed on the display as the report 60 (step S42).

The processor 301 determines whether or not there is a request for feedback of self-evaluation on the basis of the user operation (step S43). When it is determined that there is a feedback request (YES in step S43), the processor 301 executes a feedback processing routine R17.

When it is determined that there is no feedback request (NO in step S43), the processor 301 determines whether comparison of the determination value 43 between the target meeting and the meeting held in the past is requested on the basis of the user operation (step S44). When it is determined that the request for the comparison has not been made (NO in step S44), the process ends, or when it is determined that the request for the comparison has been made (YES in step S44), the processor 301 executes a comparison routine R18 of result information of the meeting, and then ends the process.

When it is determined that there is a feedback request (YES in step S43), the processor 301 executes the feedback processing routine R17, and then ends the process.

(d3-2. Feedback Processing Routine: R17)

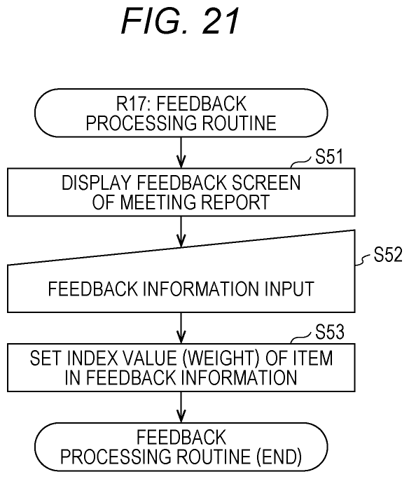

FIG. 21 is a flowchart of the feedback processing routine R17 of FIG. 20. Referring to FIG. 21, the processor 301 causes the UI screen of FIG. 26 to be displayed on the display as the FB input unit 403 in order to receive the FB information 52 of the report 60 of the target meeting from the user (step S51). The processor 301 receives the FB information 52 based on the user operation on the UI screen as the FB processing unit 405 (step S52), and the processor 301 sets the index value corresponding to the item indicated by the FB information 52 in each item corresponding to the target meeting of the determination index value DB 331 as the index value changer 418 (step S53).

(d3-3. Comparison Routine of Result Information of Meeting: R18)

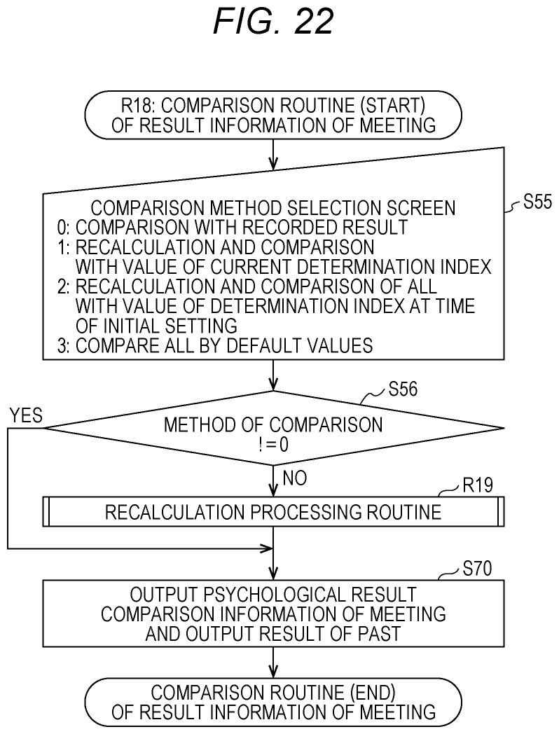

FIG. 22 is a flowchart of a comparison routine R18 of the result information of the meeting in FIG. 20. Referring to FIG. 22, the processor 301 causes a UI screen for allowing the user to select a comparison method to be displayed on the display (step S55). Options of the comparison method include: 0: comparison with the recorded result; 1: recalculation and comparison with the value of the current determination index; 2: recalculation and comparison of all with the value of the determination index at the time of initial setting execution; and 3: comparison with the default value.

The processor 301 determines an option selected on the basis of a user operation (step S56). When it is determined that 0 is selected as an option (YES in step S56), the processor 301 causes the screen of the comparison result data 271 in FIG. 27 to be displayed on the display as the comparison processing unit 406 and the comparison result outputter 404 (step S70).

On the other hand, when it is determined that an option other than 0 is selected (NO in step S56), the processor 301 executes a recalculation processing routine R19. Thereafter, in step S70, the screen of the comparison result data 271 based on a result of the recalculation processing is displayed on the display (step S70).

(Recalculation processing routine: R19)

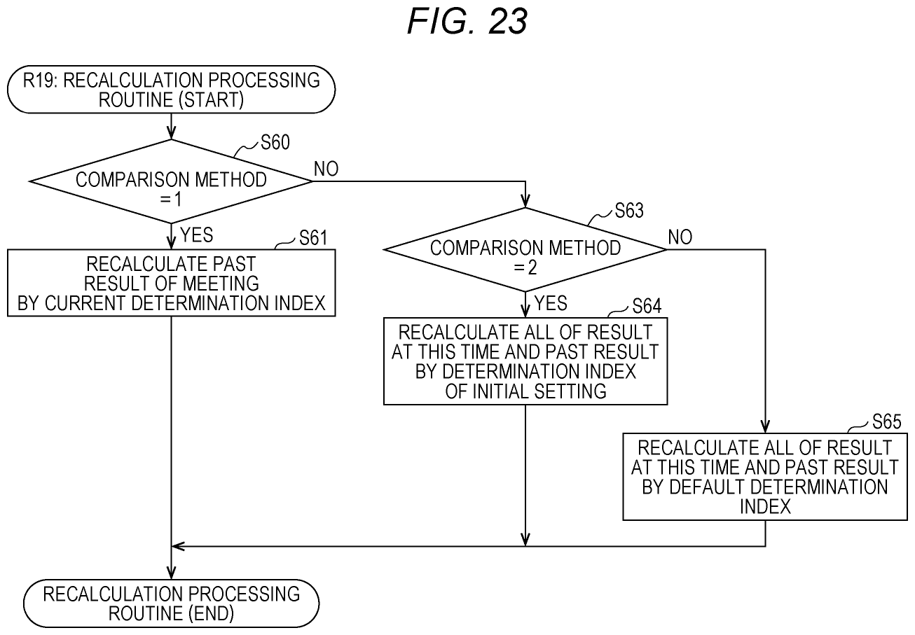

FIG. 23 is a flowchart of the recalculation processing routine R19 of FIG. 22. Referring to FIG. 23, the processor 301 determines whether the option selected by the user is 1, 2, or 3 (steps S60 and S63).

When it is determined that the option is 1 (YES in step S60), as the recalculation processing unit 407, the processor 301 retrieves the estimation value 42 of each item for each of all the meetings held this time and in the past regarding the target meeting from the measurement result management DB 334, recalculates the determination value 43 on the basis of the retrieved estimation value 42 of each item and the current index value of the item, and outputs the comparison result data 271 based on a recalculation result (step S61). For example, in a case where the target meeting is an idea development meeting and the current index value of the item (5) is changed to 3.5 by using the index value changer 418, the comparison result data 271 which is the result recalculated in step S61 is displayed like the UI screen in FIG. 28, for example.

When it is determined that the option is 2 (NO in step S60 and YES in step S63), as the recalculation processing unit 407, the processor 301 recalculates the determination value 43 on the basis of the estimation value 42 of each item and the index value used for the first evaluation of the item for each of all the above-described meetings, and outputs the comparison result data 271 based on a result of the recalculation (step S64).

When it is determined that the option is 3 (NO in step S60 and NO in step S63), as the recalculation processing unit 407, the processor 301 recalculates the determination value 43 on the basis of the estimation value 42 of each item and the default index value of the item for each of all the above-described meetings, and outputs the comparison result data 271 based on a result of the recalculation (step S65).

As described above, in the recalculation processing routine: R19, the recalculation processing unit 407 forms a redeterminer that derives the determination value 43 using a new index value. More specifically, the recalculation processing unit 407 determines the likelihood that the member has the psychological safety during the meeting on the basis of the likelihood indicated by the estimation value 42 estimated by the psychological state measurement unit 413 and a predetermined index value for at least one of a plurality of meetings (this meeting has an idea development meeting of the same purpose, and the participating members are almost the same) held at different times as illustrated in FIG. 27. The predetermined index value includes the index value after change that has been changed by the index value changer 418, a default index value, or the like.

The embodiment described above is not limited to the application of the online meeting, and may be, for example, a meeting performed face-to-face. In this case, the camera 13 and the microphone 14 may be provided in the room.

<E. Program>

In the present embodiment, a program for causing the processor 301 to execute the above-described processing is provided. Such a program includes at least a program for performing processing according to the flowcharts of FIGS. 14 to 23. Such a program can also be provided as a program product by being recorded on a computer-readable storage medium such as an optical disk 307A, a flexible disk, a compact disk-read only memory (CD-ROM), a ROM, a RAM, and a memory card attached to the computer of the information processing device 300. Alternatively, the program can be provided by being recorded as a program in the storage device 310 built in the computer. Furthermore, the program can also be provided by being downloaded via the network 100. The program may be executed by one or more processors such as the processor 301, or by a combination of a processor and a circuit such as an ASIC or an FPGA.

Note that the program may call a necessary module in a predetermined array at a predetermined timing among program modules provided as a part of the OS of the computer, and cause the processor to execute processing. In that case, the program itself does not include the module, and the processing is executed in cooperation with the OS. A program not including such a module can also be included in the program of each embodiment.

In addition, the program according to the present embodiment may be provided by being incorporated in a part of another program. Also in this case, the program itself does not include a module included in the other program, and causes the processor to execute processing in cooperation with the other program. A program incorporated in such another program can also be included in the program according to the present embodiment.

Although embodiments of the present invention have been described and illustrated in detail, the disclosed embodiments are made for purposes of illustration and example only and not limitation. The scope of the present invention should be interpreted by terms of the appended claims, and it is intended that all modifications are included in the meaning and scope equivalent to the claims

What is claimed is:

1. An information processing device that determines a psychological state of a member in a meeting, the information processing device comprising:
a voice processor that extracts a predetermined feature amount from a voice of a part corresponding to a speech of the member among voices collected during the meeting by a microphone;
an estimator that receives an input of a feature amount and estimates a likelihood that the member is in a predetermined psychological state; and
a determiner that determines a likelihood that the member is in the predetermined psychological state during the meeting on a basis of the likelihood estimated by the estimator and an index value according to a purpose of the meeting.

2. The information processing device according to claim 1, further comprising an image analyzer that extracts an image feature amount representing a predetermined action from an image of the member obtained by capturing by a camera during the meeting, wherein
the input of the feature amount received by the estimator further includes an input of the image feature amount.

3. The information processing device according to claim 1, wherein the estimator receives an input of the feature amount and estimates a likelihood that the member is in the predetermined psychological state for each of a plurality of items.

4. The information processing device according to claim 3, wherein the index value is set for each of the plurality of items.

5. The information processing device according to claim 3, wherein the determiner converts magnitude of the likelihood determined for each of the plurality of items into a numerical value.

6. The information processing device according to claim 3, wherein for one or more items according to a purpose of the meeting among the plurality of items, the converted numerical value of the item is adjusted by addition or subtraction.

7. The information processing device according to claim 1, further comprises:
an outputter that outputs the likelihood determined by the determiner;
an input interface that receives a user operation on the information processing device; and
an index value changer that changes the index value, wherein
the index value changer changes the index value on a basis of the user operation received by the input interface.

8. The information processing device according to claim 7, wherein when receiving the user operation from a plurality of users, the index value changer changes the index value on a basis of the user operation.

9. The information processing device according to claim 7, wherein
the index value changer further changes the index value according to a change rule, and
the change rule includes a rule of change based on an index value for a plurality of meetings having a same purpose.

10. The information processing device according to claim 7, wherein
the outputter
outputs the likelihood determined by the determiner for each of a plurality of meetings in which the member participates, the plurality of meetings having a same purpose and held at different times.

11. The information processing device according to claim 10, further comprising a redeterminer that determines, for at least one of the plurality of meetings, a likelihood that the member is in the predetermined psychological state during the meeting on a basis of the likelihood estimated by the estimator and a predetermined index value.

12. The information processing device according to claim 11, wherein the predetermined index value includes an index value after change that has been changed by the index value changer.

13. The information processing device according to claim 11, wherein the predetermined index value includes a default value.

14. The information processing device according to claim 7, wherein the index value changer further changes the index value on a basis of an attribute of a member of the meeting.

15. The information processing device according to claim 14, wherein when there is a predetermined number of members having a predetermined attribute among members of the meeting, the index value changer further changes the index value on a basis of the attribute.

16. The information processing device according to claim 1, wherein the purpose of the meeting is acquired from information registered regarding the meeting.

17. The information processing device according to claim 1, wherein the purpose of the meeting is acquired by performing recognition processing on voices collected during the meeting.

18. The information processing device according to claim 7, wherein the outputter outputs information regarding the index value used by the determiner to determine the likelihood.

19. A non-transitory recording medium storing a computer readable program for causing a computer to execute a method for determining a psychological state of a member in a meeting, wherein
the method comprises:
extracting a predetermined feature amount from a voice of a part corresponding to a speech of the member among voices collected during the meeting by a microphone;
receiving an input of a feature amount and estimating a likelihood that the member is in a predetermined psychological state; and
determining a likelihood that the member is in the predetermined psychological state during the meeting on a basis of the likelihood estimated and an index value according to a purpose of the meeting.
